(19) United States
(12) Patent Application Publication
(10) Pub. No.: US 2013/0123184 A1
(43) Pub. Date: May 16, 2013

(54) METHODS FOR INCREASING LEVELS OF HUMAN FETAL HEMOGLOBIN

(71) Applicants: Mitchell KEEGAN, Cambridge, MA (US); Janelle R. KEYS, St. Lucia (AU); Richard L. PIEKARZ, Silver Spring Drive, MD (US); Susan E. BATES, Bethesda, MD (US)

(72) Inventors: Mitchell KEEGAN, Cambridge, MA (US); Janelle R. KEYS, St. Lucia (AU); Richard L. PIEKARZ, Silver Spring Drive, MD (US); Susan E. BATES, Bethesda, MD (US)

(73) Assignees: National Institutes of Health, Rockville, MD (US); Celgene Corporation, Summit, NJ (US)

(21) Appl. No.: 13/722,995

(22) Filed: Dec. 20, 2012

Related U.S. Application Data
(63) Continuation of application No. 12/523,565, filed on Jun. 11, 2010, now abandoned.

(51) Int. Cl. A61K 38/12

(52) U.S. Cl. CPC A61K 38/12
USPC 514/15.3; 435/375

(57) ABSTRACT

The invention provides methods for increasing the level of human fetal hemoglobin in a subject or cell in need thereof. The methods can be used with subjects suffering from a β-chain hemoglobinopathy including thalassemia (e.g., β-thalassemia) or sickle cell anemia.

### BACKGROUND OF THE INVENTION

Hemoglobin is a critically important molecule as it carries oxygen to our tissues. In adults, hemoglobin is predominantly composed of two pairs of protein chains—α-globin chains and β-globin chains. Inherited mutations of the β-globin locus cause β-chain hemoglobinopathies such as thalassemia and sickle cell anemia which are serious world wide health burdens and the most common genetic disorders in the world.

Within the human genome the β-globin locus contains 5β-like genes (ε, Gγ, Aγ, δ and β) which are expressed sequentially during haematopoietic development. During primitive erythropoiesis, the embryonic ε-globin gene is expressed in nucleated, yolk-sac derived erythroid cells. Later, during fetal definitive erythropoiesis, the tandem fetal γ-globin genes are expressed in enucleated erythroid cells of the fetal liver to yield fetal hemoglobin. Finally, the β-globin gene, and to a much lesser extent the δ-globin gene, are expressed initially in the liver during fetal definitive erythropoiesis and ultimately in bone marrow-derived erythroid cells during adult definitive erythropoiesis.

It has been suggested that reactivation of the fetal specific γ-globin genes in the adult bone marrow would treat β-chain hemoglobinopathies, as it does when there is coinheritance of mutations which cause the disease hereditary persistence of fetal hemoglobin (HPFH), e.g., see Noguchi et al., N. Eng. J. Med. 318:96, 1988; Charache et al., Blood 69:109, 1987; Reed et al., Blood 25:37, 1965; Goldberg et al., J. Biol. Chem. 252:3414, 1977; Reich et al., Blood 96:3357, 2000. One of the ultimate aims of globin research worldwide is therefore to specifically reactivate fetal γ-globin gene expression, in an attempt to treat β-chain hemoglobinopathies such as thalassemia and sickle cell anemia.

### SUMMARY OF THE INVENTION

In one aspect, the present invention provides methods for increasing the level of fetal hemoglobin in a subject in need thereof. Such methods involve administering a therapeutically effective amount of romidepsin to a subject in need thereof.

Romidepsin is a natural product which was isolated from Chromobacterium violaceum by Fujisawa Pharmaceuticals. See Published Japanese Patent Application Hei 7 (1995)-64872; U.S. Pat. No. 4,977,138. It is a bicyclic peptide consisting of four amino acid residues (D-valine, D-cysteine, dehydrobutyrine, and L-valine) and a novel acid (3-hydroxy-7-mercapto-4-heptenoic acid). Romidepsin is a depsipeptide which contains both amide and ester bonds. In addition to fermentation from C. violaceum, romidepsin can also be prepared by semi-synthesis or total synthesis. The total synthesis of romidepsin reported by Kahn et al. involves 14 steps and yields romidepsin in 18% overall yield. J. Am. Chem. Soc. 118:7237-7238, 1996. In one embodiment, the administered romidepsin is of the formula:

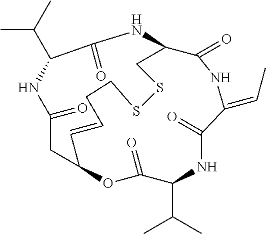 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

In certain embodiments, the subject to which romidepsin is administered has a β-chain hemoglobinopathy. For example, the subject may have thalassemia (e.g., β-thalassemia) or sickle cell anemia.

In another aspect, the invention provides methods of increasing the level of human fetal hemoglobin in cells by contacting cells with an amount of romidepsin effective to increase the level of human fetal hemoglobin. In certain embodiments, the cells are cells that produce hemoglobin, e.g., erythrocytes and erythroid progenitors.

### DEFINITIONS

Definitions of other terms used throughout the specification include:

As used herein and in the appended claims, the singular forms “a”, “an”, and “the” include the plural reference unless the context clearly indicates otherwise. Thus, for example, a reference to “a cell” includes a plurality of such cells.

“Animal”: As used herein, the term “animal” refers to any member of the animal kingdom. In some embodiments, animals include, but are not limited to, mammals, birds, reptiles, amphibians, fish, and/or worms. In some embodiments, “animal” refers to a human, at any stage of development. In some embodiments, “animal” refers to a non-human animal, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, an animal may be a transgenic animal, genetically-engineered animal, and/or clone.

“Depsipeptide”: The term “depsipeptide”, as used herein, refers to peptides that contain both ester and amide bonds. Naturally occurring depsipeptides are usual cyclic. Some depsipeptides have been shown to have potent antibiotic activity. Examples of depsipeptides include actinomycin, enniatins, valinomycin, and romidepsin.

“Effective amount”: In general, the “effective amount” of an active agent or combination of agents refers to an amount sufficient to elicit the desired biological response. As will be appreciated by those of ordinary skill in this art, the effective amount of active agent may vary depending on such factors as the desired biological endpoint, the pharmacokinetics of the agent(s) being delivered, the disease being treated, the route and schedule of administration, and the subject. In general, an effective amount of romidepsin in the methods of the invention is an amount that results in an increase in the level of fetal hemoglobin. In certain embodiments, an effective amount of romidepsin in the methods of the invention is the amount that causes an improvement in the symptoms of a subject suffering from a β-chain hemoglobinopathy, e.g., thalassemia or sickle cell anemia.

“Peptide” or “protein”: According to the present invention, a “peptide” or “protein” comprises a string of at least three amino acids linked together by peptide bonds. The terms “protein” and “peptide” may be used interchangeably. Peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In certain embodiments, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide. In certain embodiments, peptide refers to depsipeptide.

“Pharmaceutically acceptable salt”: The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19, 1977. The salts can be prepared in situ during the final isolation and purification of the compounds used in the inventive methods, or separately by reacting the free base functionality with a suitable organic or inorganic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, carnphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

“Pharmaceutically acceptable ester”: The term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates. In certain embodiments, the esters are cleaved by enzymes such as esterases.

“Pharmaceutically acceptable prodrug”: The term “pharmaceutically acceptable prodrug”, refers to prodrugs which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds used in the inventive methods. The term “prodrug” refers to compounds that are rapidly transformed in vivo to yield the parent compound, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987.

“Romidepsin”: The term “romidepsin”, refers to a natural product of the chemical structure:

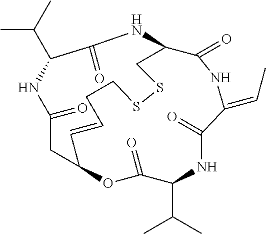 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

Romidepsin is a potent HDAC inhibitor and is also known in the art by the names FK228, FR901228, NSC630176, or depsipeptide. The identification and preparation of romidepsin is described in U.S. Pat. No. 4,977,138. The molecular formula is C₂₄H₃₆N₄O₆S₂; and the molecular weight is 540.71. Romidepsin has the chemical name, (1S,4S,10S,16E,21R)-7-[(2Z)-ethylidene]-4,21-diisopropyl-2-oxa-12,13-dithia-5,8,20,23-tetraazabicyclo[8.7.6]tricos-16-ene-3,6,9,19,22-pentanone. Romidepsin has been assigned the CAS number 128517-07-7. In crystalline form, romidepsin is typically a white to pale yellowish white crystal or crystalline powder. When reference is made to “romidepsin” herein, it will be understood that any pharmaceutically acceptable salt of romidepsin may be employed. Alternatively or additionally, romidepsin may be produced through use of a pharmaceutically acceptable pro-drug, ester, protected form or other derivative of romidepsin as described herein.

### BRIEF DESCRIPTION OF THE DRAWING

FIG. 1 shows the increase in γ globin gene expression in transgenic murine cells when treated with 0.185 nM or 1.85 nM romidepsin. β globin gene expression was not affected by either treatment.

FIG. 2 shows that when the 0.185 nM and 1.85 nM data of FIG. 1 are combined there is a 130-fold increase in y globin expression, as compared to mouse a globin expression and untreated cells (mean±SEM, n=6).

FIG. 3 shows the fetal hemoglobin levels plotted over time from initiation of romidepsin therapy for two human patients. Fetal hemoglobin levels were expressed first as a percent of the patient's total hemoglobin and then corrected (by multiplying the total hemoglobin by percent fetal hemoglobin).

### DETAILED DESCRIPTION OF CERTAIN EMBODIMENTS OF THE INVENTION

The present application refers to patent and non-patent publications. The contents of each of these publications is incorporated herein by reference.

As described in the Examples, we have demonstrated that romidepsin causes a significant increase in γ-globin gene expression (a component of fetal hemoglobin). Using fetal liver cells from transgenic mice that contained a yeast artificial chromosome with the full human β-globin locus we have shown that a 130-fold increase in γ-globin gene expression can be obtained in vitro using nM concentrations of romidepsin. Furthermore, in a human clinical trial of 44 patients, we have shown that fetal hemoglobin levels were increased greater than 2-fold in 77% of patients and greater than 10-fold in 34% of patients treated with romidepsin for at least two months.

In one aspect, the present invention provides methods for increasing the level of fetal hemoglobin in a subject in need thereof. Such methods involve administering a therapeutically effective amount of romidepsin to the subject. In general, the methods will be useful for any subject that would benefit from an increase in fetal hemoglobin levels. For example, as discussed in the Background, the methods are particularly useful for subjects suffering from β-chain hemoglobinopathies (e.g., see Noguchi et al., N. Eng. J. Med. 318:96, 1988; Charache et al., Blood 69:109, 1987; Reed et al., Blood 25:37, 1965; Goldberg et al., J. Biol. Chem. 252:3414, 1977; Reich et al., Blood 96:3357, 2000). In certain embodiments, the methods are used with a subject suffering from thalassemia, e.g., β-thalassemia. In other embodiments, the methods are used with a patient suffering from sickle cell anemia. The subject can bean animal, preferably a human.

In another aspect, the present invention provides methods of increasing the level of human fetal hemoglobin in cells by contacting cells with an amount of romidepsin effective to increase the level of human fetal hemoglobin. In certain embodiments, the cells are cells that produce hemoglobin, e.g., erythrocytes and erythroid progenitors. In one embodiment, the cells are contacted with a concentration of romidepsin that ranges from approximately 0.0185 nM to approximately 18.5 nM. For example, the concentration may range from 0.185 nM to approximately 1.85 nM. The contacted cell may be in culture or within an animal, e.g., a human.

### Romidepsin

Romidepsin is a cyclic depsipeptide of formula:

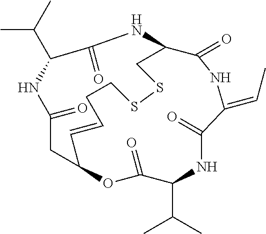 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

The inventive methods may use salts, esters, pro-drugs, isomers, stereoisomers (e.g., enantiomers, diastereomers), tautomers, derivatives of romidepsin, or combinations of these. In certain embodiments, the romidepsin used is pharmaceutical grade material and meets the standards of the U.S. Pharmacopoeia, Japanese Pharmacopoeia, or European Pharmacopoeia. In certain embodiments, the romidepsin is at least 95%, at least 98%, or at least 99% pure. In certain embodiments, the romidepsin is at least 95%, at least 98%, or at least 99% monomeric. In certain embodiments, no impurities are detectable in the romidepsin materials (e.g., oxidized material, reduced material, dirnerized or oligomerized material, side products, etc.).

The inventive methods may also use a derivative of romidepsin. In certain embodiments, the derivative of romidepsin is of the formula (I):

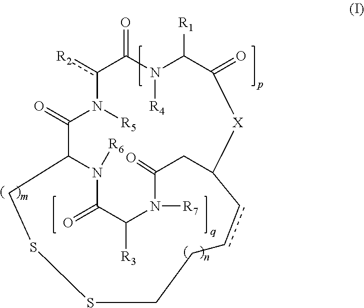

wherein

m is 1, 2, 3 or 4;

n is 0, 1, 2 or 3;

p and q are independently 1 or 2;

X is O, NH, or NR₈;

R₁, R₂, and R₃ are independently hydrogen; unsubstituted or substituted, branched or unbranched, cyclic or acyclic aliphatic; unsubstituted or substituted, branched or unbranched, cyclic or acyclic heteroaliphatic; unsubstituted or substituted aryl; or unsubstituted or substituted heteroaryl;

R₄, R₅, R₆, R₇ and R₈ are independently hydrogen; or substituted or unsubstituted, branched or unbranched, cyclic or acyclic aliphatic; and pharmaceutically acceptable salts thereof. In certain embodiments, m is 1. In certain embodiments, n is 1. In certain embodiments, p is 1. In certain embodiments, q is 1. In certain embodiments, X is O. In certain embodiments, R₁, R₂, and R₃ are unsubstituted, or substituted, branched or unbranched, acyclic aliphatic. In certain embodiments, R₄, R₅, R₆, and R₇ are all hydrogen.

In certain embodiments, the derivative of romidepsin is of the formula (II):

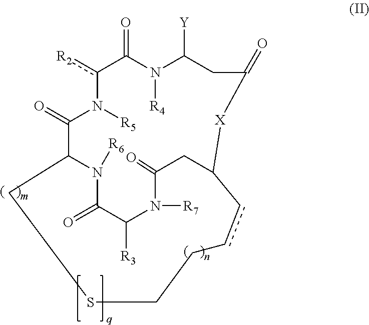

wherein:

m is 1, 2, 3 or 4;

n is 0, 1, 2 or 3;

q is 2 or 3;

X is O, NH, or NR₈;

Y is OR₈, or SR₈;

R₂ and R₃ are independently hydrogen; unsubstituted or substituted, branched or unbranched, cyclic or acyclic aliphatic; unsubstituted or substituted, branched or unbranched, cyclic or acylic heteroaliphatic; unsubstituted or substituted aryl; or unsubstituted or substituted heteroaryl;

R₄, R₅, R₆, R₇ and R₈ are independently selected from hydrogen; or substituted or unsubstituted, branched or unbranched, cyclic or acyclic aliphatic; and pharmaceutically acceptable salts thereof. In certain embodiments, m is 1. In certain embodiments, n is 1. In certain embodiments, q is 2. In certain embodiments, X is O. In other embodiments, X is NH. In certain embodiments, R₂ and R₃ are unsubstituted or substituted, branched or unbranched, acyclic aliphatic. In certain embodiments, R₄, R₅, R₆, and R₇ are all hydrogen.

In certain embodiments, the derivative of romidepsin is of the formula (III):

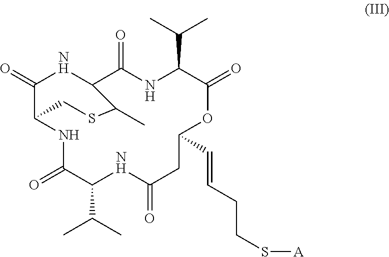

wherein A is a moiety that is cleaved under physiological conditions to yield a thiol group and includes, for example, an aliphatic or aromatic acyl moiety (to form a thioester bond); an aliphatic or aromatic thioxy (to form a disulfide bond); or the like; and racemates, enantiomers, isomers, tautomers, salts, esters, and prodrugs thereof. Such aliphatic or aromatic groups can include a substituted or unsubstituted, branched or unbranched, cyclic or acyclic aliphatic group; a substituted or unsubstituted aromatic group; a substituted or unsubstituted heteroaromatic group; or a substituted or unsubstituted heterocyclic group. A can be, for example, —COR₁, —SC(═O)—O—R₁, or —SR₂. R₁ is independently hydrogen; substituted or unsubstituted amino; substituted or unsubstituted, branched or unbranched, cyclic or acyclic aliphatic; substituted or unsubstituted aromatic group; substituted or unsubstituted heteroaromatic group; or a substituted or unsubstituted heterocyclic group. In certain embodiment, R₁ is hydrogen, methyl, ethyl, n-propyl, iso-propyl, n-butyl, isobutyl, benzyl, or bromobenzyl. R₂ is a substituted or unsubstituted, branched or unbranched, cyclic or acyclic aliphatic group; a substituted or unsubstituted aromatic group; a substituted or unsubstituted heteroaromatic group; or a substituted or unsubstituted heterocyclic group. In certain embodiments, R₂ is methyl, ethyl, 2-hydroxyethyl, isobutyl, fatty acids, a substituted or unsubstituted benzyl, a substituted or unsubstituted aryl, cysteine, homocysteine, or glutathione.

Processes for preparing romidepsin are known in the art. Since romidepsin is a natural product, it is typically prepared by isolation from a fermentation of a microorganism that produces it. In certain embodiments, romidepsin or a derivate thereof is purified from a fermentation, for example, of Chromobacterium violaceum. See, e.g., Ueda et al., J. Antibiot. (Tokyo) 47:301-310, 1994; Nakajima et al., Exp. Cell Res. 241:126-133, 1998; WO 02/20817; U.S. Pat. No. 4,977,138. In other embodiments, romidepsin or a derivative thereof is prepared by semi-synthesis or total synthesis. J. Am. Chem. Soc. 118:7237-7238, 1996.

### Administration

Romidepsin may be administered via any route and schedule that delivers a therapeutically effective amount to the subject. In certain embodiments, romidepsin is administered orally. When administered orally, romidepsin may be given several times a day, once daily, twice weekly, once weekly, etc. In certain embodiments, romidepsin is administered intravenously. In certain embodiments, romidepsin is administered intravenously over a 1-6 hour time frame. In certain particular embodiments, romidepsin is administered intravenously over 3-4 hours. In certain particular embodiments, romidepsin is administered intravenously over 5-6 hours. In certain embodiments, romidepsin is administered intravenously one day followed by several days in which romidepsin is not administered. In certain embodiments, romidepsin is administered intravenously twice a week. In certain embodiments, romidepsin is administered intravenously once a week. In other embodiments, romidepsin is administered intravenously every other week. When given intravenously, romidepsin may be administered continuously or in cycles. For example, in certain embodiments, romidepsin is administered intravenously on days 1, 8, and 15 of a 28 day cycle. The 28 day cycle may be repeated. In certain embodiments, the 28 day cycle is repeated 3-10 times. In certain embodiments, the treatment includes 5 cycles. In certain embodiments, the treatment includes 6 cycles. In certain embodiments, the treatment includes 7 cycles. In certain embodiments, the treatment includes 8 cycles. In certain embodiments, greater than 10 cycles are administered. In general, treatment will continue as long as the subject is responding. In certain embodiments, therapy may be terminated once there is disease progression, a cure or remission is achieved, or side effects become intolerable.

The therapeutically effective amount of romidepsin administered may vary depending on the subject, the disease being treated, the dosage form and the route and schedule of administration. In certain embodiments, the romidepsin is dosed in the range of 1 mg/m² to 150 mg/m². When administered intravenously, lower dosages may be used, e.g., 1 mg/m² to 50 mg/m² or 5 mg/m² to 25 mg/m². In other embodiments, the dosage ranges from 10 mg/m² to 20 mg/m². In certain embodiments, the dosage ranges from 5 mg/m² to 10 mg/m². In other embodiments, the dosage ranges from 10 mg/m² to 15 mg/m². In still other embodiments, the dosage is approximately 12 mg/m². In still other embodiments, the dosage is approximately 13 mg/m². In still other embodiments, the dosage is approximately 14 mg/m². In still other embodiments, the dosage is approximately 15 mg/m². When administered orally, higher dosages may be used because of lower bioavailability. For example, in certain embodiments, a dosage from 25 mg/m² to 150 mg/m² or 75 mg/m² to 100 mg/m² may be used.

Romidepsin is known to have anti-proliferative effects, e.g., for treating certain cancers. In certain embodiments, romidepsin is administered at dosage levels that do not cause these anti-proliferative effects. For example, a dosage within the range of 1 mg/m² to 10 mg/m², 1 mg/m² to 5 mg/m², or 5 mg/m² to 10 mg/m² may satisfy these criteria.

### Pharmaceutical Compositions

In certain embodiments, the inventive methods may involve administering romidepsin in the context of a pharmaceutical composition that further includes inter alia a pharmaceutically acceptable carrier.

As used herein, a “pharmaceutically acceptable carrier” includes any and all solvents, diluents, or other liquid vehicles, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with romidepsin or another pharmaceutical agent present in the composition, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; Cremophor; Solutol; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. When administered orally, it may prove advantageous to administer romidepsin in combination with a permeation enhancer or other agent that improves oral bioavailability. The permeation enhancer may be included within the same pharmaceutical composition as romidepsin or administered separately.

In certain embodiments, romidepsin may be administered as a sustained release pharmaceutical composition. As is known in the art, these composition typically include a hydrophobic coating that delays the release of the pharmaceutical agent in vivo. Suitable coatings are described in the art, e.g., see Remington's Pharmaceutical Sciences, Fifteenth Edition, E. W. Martin (Mack Publishing Co., Easton, Pa., 1975).

It will also be appreciated that romidepsin can exist in free form for administration, or where appropriate, as a pharmaceutically acceptable salt, ester or any other adduct or derivative which upon administration to a subject in need is capable of providing, directly or indirectly, a compound as otherwise described herein, or a metabolite or residue thereof, e.g., a prodrug.

### Combinations

Romidepsin may be administered in combination with other pharmaceutical agents. In particular, romidepsin may be administered in combination with another agent that increases levels of fetal hemoglobin (e.g., 5-azacytidine, hydroxyurea, a butyrate, one or more of the short chain fatty acids described in Liakopoulou et al., Blood 86:3227, 1995, etc.). In another embodiment, romidepsin may be administered in combination with another histone deacetylase (HDAC) inhibitor. For example, the pharmaceutical composition might include one more of a butyrate (e.g., sodium butyrate, arginine butyrate, sodium phenylbutyrate, etc.), HC-toxin, trichostatin, MS-275, apicidin, and derivatives of butyryl hydroxamic (B—H) acid, propionyl hydroxamic (P—H) acid, subericbis hydroxamic acid (SBHA), suberoylanilide hydroxamic acid (SAHA), etc. In certain embodiments, the composition does not include another HDAC inhibitor besides romidepsin.

In other embodiments, pharmaceutical compositions for use in accordance with the present invention further comprise an anti-inflammatory agent such as aspirin, ibuprofen, acetaminophen, etc., pain reliever, anti-nausea medication, or anti-pyretic. In certain embodiments, such compositions comprise an agent to treat gastrointestinal disturbances such as nausea, vomiting, and diarrhea. These additional agents may include anti-emetics, anti-diarrheals, fluid replacement, electrolyte replacement, etc. In other embodiments, such compositions comprise electrolyte replacement or supplementation such as potassium, magnesium, and calcium, in particular, potassium and magnesium. In certain embodiments, such compositions include an anti-arrhythmic agent. In certain embodiments, the compositions comprise a platelet booster, for example, an agent that increases the production of platelets. In certain embodiments, the compositions comprise an agent to boost the production of blood cells such as erythropoietin. In certain embodiments, the compositions further comprise an agent to prevent hyperglycemia. It will also be appreciated that the methods may be combined with any known method for treating n-chain hemoglobinopathies (e.g., blood transfusions, chelation therapy, etc.).

### EXAMPLES

These and other aspects of the present invention will be further appreciated upon consideration of the following Examples, which are intended to illustrate certain particular embodiments of the invention but are not intended to limit its scope, as defined by the claims.

### Example 1

### Romidepsin Increases Fetal Hemoglobin Levels in Transgenic Mice Cells

Experiments were performed with mice that are transgenic for a yeast artificial chromosome that contains the full human β-globin locus. Expression of the human locus is required as mice do not undergo a specific fetal-to-adult globin switch. However mice do have the appropriate transcription factors to interpret and express the human globin locus.

To determine if romidepsin alters human globin expression erythroid colony forming unit assays were conducted. This involved extracting fetal liver cells from the developing embryo, at a time when both γ and β globin are expressed. Cells were then cultured in a semi-solid media (methyl-cellulose) containing growth factors (SCF and Epo) with or without romidepsin. We harvested CFUe colonies at 2 days of culture (a pool of 10 from each treatment, each treatment was performed in duplicate) and performed an immediate cell lysis and cDNA amplification. The cDNA was then subjected to real-time PCR to determine levels of mouse α, human γ and human β globin gene expression. The cells were treated with a range of romidepsin concentrations from 0.185 nM to 18.5 nM, and the colony assay was performed on 6 separate occasions.

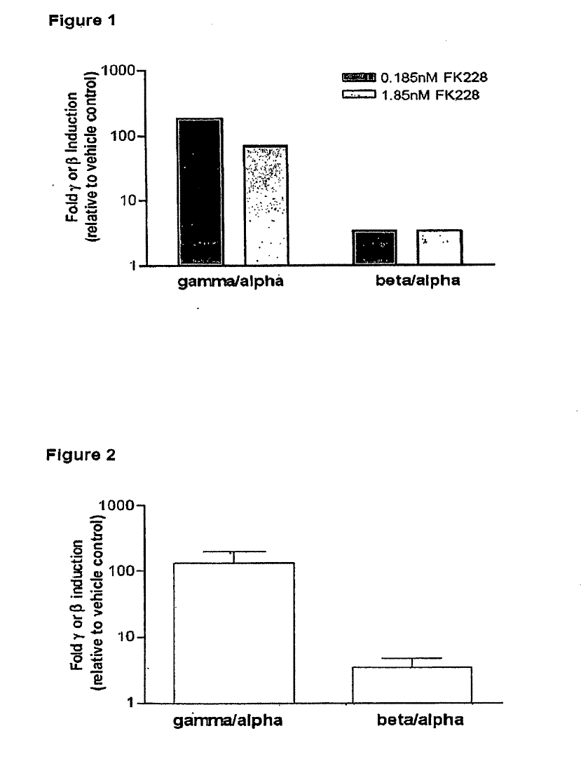

The results (0.185 nM or 1.85 nM romidepsin) are presented in FIG. 1 relative to mouse α levels, and relative to the control performed in each particular experiment. In general it was found that there was an up-regulation of γ globin gene expression following romidepsin treatment and no change in β globin gene expression. When the 0.185 nM and 1.85 nM data are combined there is a 130-fold increase in γ globin expression, as compared to mouse α globin expression and untreated cells. This data is presented in FIG. 2 (mean±SEM, n=6).

Each colony was also analyzed for toxicity effects. Although accurate data was not gathered it was found that a dose of 18.5 nM was toxic to the fetal liver cells. Thus globin analysis at this dose is not presented. At 0.185 nM and 1.85 nM no differences in either total number of colonies, or in the size of CFUe were detected. Thus romidepsin does not display unwanted anti- or pro-proliferative activities at these concentrations.

There was some variation between experiments and doses tested. This variability may be due to many factors including the quality of colonies generated in each experiment. In addition, the mouse model of human globin expression is sometimes difficult to interpret because the levels of human globin are relatively low as compared to murine levels. In addition, it was noted that expression of human globin was lower at the highest doses of romidepsin. This may be due to potential toxic effects however it could also have been due to the variability that was observed. On occasion, a decrease in β globin expression was observed however this was not consistent.

### Example 2

### Romidepsin Increases Fetal Hemoglobin Levels in Humans

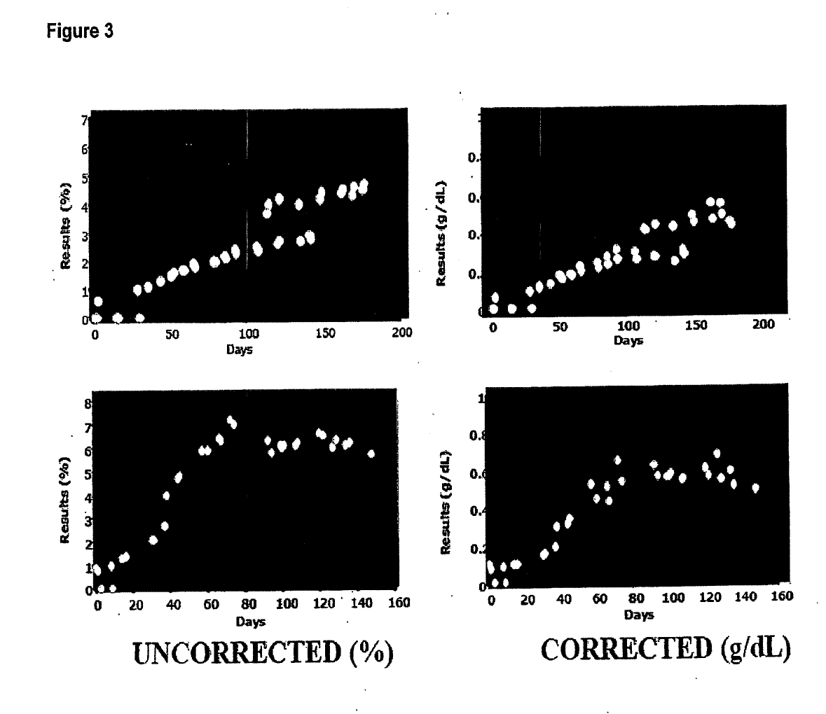

As part of an ongoing phase II clinical trial into the effects of romidepsin in patients with cutaneous (CTCL) and peripheral (PTCL) T-cell lymphoma, the levels of circulating fetal hemoglobin were determined. From a total of 53 patients in the trial, 44 were evaluated. Romidepsin was administered as a 4 hour infusion on days 1, 8, and 15 of a 28 day cycle with a dose of 14 mg/m². Fetal hemoglobin levels were measured during at least two months of treatment with romidepsin. Fetal hemoglobin levels were expressed first as a percent of the patient's total hemoglobin and then corrected (by multiplying the total hemoglobin by percent fetal hemoglobin). FIG. 3 shows the fetal hemoglobin levels plotted over time from initiation of therapy for two patients. No patients enrolled had a known hemoglobinopathy. The results after at least two months of romidepsin therapy are summarized in Tables 1 and 2.

TABLE 1

| Fold increase in fetal hemoglobin | Number of patients | % of patients |
|---|---|---|
| 0-2 | 10 | 23% |
| 2-5 | 10 | 23% |
| 5-10 | 9 | 20% |
| 10-20 | 10 | 23% |
| >20 | 5 | 11% |

TABLE 2

| Fold increase in fetal hemoglobin | Number of patients | % of patients |
|---|---|---|
| 0-2 | 10 | 23% |
| >2 | 34 | 77% |
| >5 | 24 | 55% |
| >10 | 15 | 34% |
| >20 | 5 | 11% |

As shown in Table 2, fetal hemoglobin levels were increased greater than 2-fold in 77% of patients and greater than 10-fold in 34% of patients treated with romidepsin for at least two months.

### Equivalents and Scope

The foregoing has been a description of certain non-limiting embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a,”, “an” and “the” may mean one or more than one unless indicated to the contrary or otherwise evident from the context. Claims or descriptions that include “of” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

What is claimed is:

1. A method of increasing the level of human fetal hemoglobin in a subject, the method comprising:
administering a therapeutically effective amount of romidepsin to a subject in need of increased levels of human fetal hemoglobin.

2. The method of claim 1, wherein romidepsin is of the formula:

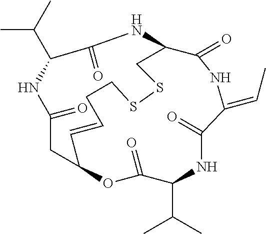 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

3. The method of claim 1, wherein the subject has a β-chain hemoglobinopathy.

4. The method of claim 1, wherein the subject has thalassemia.

5. The method of claim 1, wherein the subject has sickle cell anemia.

6. The method of claim 1, wherein the romidepsin is administered orally.

7. The method of claim 1, wherein the romidepsin is administered intravenously.

8. The method of claim 1, wherein the therapeutically effective amount of romidepsin ranges from 1 mg/m² to 150 mg/m².

9. The method of claim 6, wherein the therapeutically effective amount of romidepsin ranges from 75 mg/m² to 100 mg/m².

10. The method of claim 7, wherein the therapeutically effective amount of romidepsin ranges from 5 mg/m² to 25 mg/m².

11. The method of claim 10, wherein the therapeutically effective amount of romidepsin ranges from 10 mg/m² to 20 mg/m².

12. The method of claim 11, wherein the therapeutically effective amount of romidepsin is 14 mg/m².

13. The method of claim 1, wherein the subject is a human.

14. A method of increasing the level of human fetal hemoglobin in a cell, the method comprising:
administering an amount of romidepsin effective to increase the level of human fetal hemoglobin in a cell.

15. The method of claim 14, wherein the cell is a cell that produces hemoglobin.

16. The method of claim 14, wherein the concentration of romidepsin ranges from approximately 0.0185 nM to approximately 18.5 nM.

17. The method of claim 14, wherein the concentration of romidepsin ranges from approximately 0.185 nM to approximately 1.85 nM.

18. The method of claim 14, wherein the cell is in culture.

19. The method of claim 14, wherein the cell is within an animal.
